(19) United States
(12) Patent Application Publication
(10) Pub. No.: US 2025/0154092 A1
(43) Pub. Date: May 15, 2025

(54) METHOD FOR PRODUCING ETHYL LACTATE

(71) Applicant: National Taiwan University, Taipei City (TW)

(72) Inventors: Chia-Wen WU, Taipei City (TW); Wei-Sheng LIAO, Taipei City (TW); Jih-Peng SUN, Taipei City (TW); Tzu-Yun PENG, Taipei City (TW)

(21) Appl. No.: 18/940,040

(22) Filed: Nov. 7, 2024

Related U.S. Application Data
(60) Provisional application No. 63/597,497, filed on Nov. 9, 2023.

(30) Foreign Application Priority Data
Jul. 11, 2024 (TW) 113125978

(51) Int. Cl. C07C 67/08

(52) U.S. Cl. CPC C07C 67/08; C07C 2523/18; C07C 2527/133

(57) ABSTRACT

A method for producing ethyl lactate includes subjecting lactic acid and ethanol to an esterification reaction in the presence of an antimony catalyst, so as to obtain ethyl lactate. The antimony catalyst is selected from the group consisting of antimony trioxide, antimony pentoxide, antimony trichloride, and combinations thereof, and the esterification reaction is carried out at a temperature ranging from 80° C. to 88° C. and under a pressure of 1 atm.

### CROSS-REFERENCE TO RELATED APPLICATION

This application claims priority to Taiwanese Invention Patent Application No. 113125978, filed on Jul. 11, 2024, and claims the benefit of U.S. Provisional Patent Application No. 63/597,497, filed on Nov. 9, 2023. The entire content of the Taiwanese and U.S. patent applications are incorporated herein by reference.

### FIELD

The disclosure relates to a method for producing ethyl lactate.

### BACKGROUND

A technique currently used for preparing ethyl lactate involves subjecting lactic acid and ethanol to an esterification reaction. Since the rate of the esterification reaction is extremely slow, a solid acid catalyst may be used to increase the rate of such esterification reaction between the lactic acid and the ethanol. At present, the solid acid catalyst commonly used in preparation of ethyl lactate may include, for instance, Amberlyst® 15, a zirconium dioxide (ZrO₂)-silicon dioxide (SiO₂) composite, a tungsten trioxide (WO₃)-silicon dioxide (SiO₂) composite, and so forth. However, the above-mentioned solid acid catalysts have problems such as high price, poor stability, or poor catalytic effect.

Thus, there is still a need for those skilled in the art to develop a new way for producing ethyl lactate more efficiently so as to solve the aforesaid problems.

### SUMMARY

Therefore, an object of the disclosure is to provide a method for producing ethyl lactate that can alleviate at least one of the drawbacks of the prior art. The method includes:
subjecting lactic acid and ethanol to an esterification reaction in the presence of an antimony catalyst, so as to obtain ethyl lactate.

The antimony catalyst is selected from the group consisting of antimony trioxide, antimony pentoxide, antimony trichloride, and combinations thereof, and the esterification reaction is carried out at a temperature ranging from 80° C. to 88° C. and under a pressure of 1 atm.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for producing ethyl lactate. The method includes:
subjecting lactic acid (also known as 2-hydroxypropionic acid) and ethanol to an esterification reaction in the presence of an antimony catalyst, so as to obtain ethyl lactate.

According to the present disclosure, the antimony catalyst is selected from the group consisting of antimony trioxide (Sb₂O₃), antimony pentoxide (Sb₂O₅·nH₂O), antimony trichloride (SbCl₃), and combinations thereof, and the esterification reaction is carried out at a temperature ranging from 80° C. to 88° C. and under a pressure of 1 atm. By virtue of restricting selection of the antimony catalyst to the aforesaid group and performing the esterification reaction under certain conditions (i.e., the aforesaid temperature and pressure), the antimony catalyst exhibits a good catalytic effect on the esterification reaction of the lactic acid and the ethanol, thereby enabling the ethyl lactate to be produced efficiently.

In certain embodiments, a weight ratio of the antimony catalyst to the lactic acid may range from 0.01 to 0.06 in decimal form. To be specific, compared with a conventional solid acid catalyst such as Amberlyst® 15, a respective one of antimony trioxide, antimony pentoxide, and antimony trichloride is cheaper and has better thermal stability.

In certain embodiments, the lactic acid may have a purity of not lower than 90%. In an exemplary embodiment, the purity of the lactic acid is 90%.

In certain embodiments, a volume ratio of the ethanol to the lactic acid may range from 1.41 to 5.64 in decimal form. Specifically, the lactic acid is present in an amount ranging from 5 parts by volume to 10 parts by volume, and the ethanol is present in an amount ranging from 14.1 parts by volume to 28.2 parts by volume.

According to the present disclosure, a time period for carrying out the esterification reaction may be adjusted according to practical requirements. For example, the esterification reaction may be carried out for 1 hour, 3 hours, or 6 hours, or longer period of time. In an exemplary embodiment, the esterification reaction is carried out for the time period ranging from 3 hours to 6 hours, which enables a relatively high yield of the ethyl lactate to be obtained.

In the method for producing ethyl lactate according to the present disclosure, by virtue of selecting certain types of the antimony catalyst that is cheap and having good stability (i.e., antimony trioxide, antimony pentoxide, antimony trichloride, and combinations thereof), and by setting certain conditions for conducting the esterification reaction (i.e., the temperature ranging from 80° C. to 88° C. and the pressure of 1 atm), the ethyl lactate can be produced efficiently.

The present disclosure will be further described by way of the following examples. However, it should be understood that the following examples are intended solely for the purpose of illustration and should not be construed as limiting the present disclosure in practice.

### Examples A-1 to A-3

### Example A-1 (EA-1)

The method for producing ethyl lactate of the present disclosure was conducted as follows. First, 163 mg of antimony trioxide (Sb₂O₃; serving as an antimony catalyst), 5 mL of lactic acid (obtained from National Atomic Research Institute; purity: 90%), and 14.1 mL of anhydrous ethanol were mixed to obtain a first mixture, followed by subjecting the first mixture to an esterification reaction, which was carried out at a temperature of 80° C. and under a pressure of 1 atm for 1 hour, so as to form ethyl lactate, thereby obtaining a second mixture containing the ethyl lactate and the antimony trioxide. Subsequently, the second mixture was subjected to filtration using a syringe filter, so as to remove the antimony trioxide, thereby obtaining a filtrate containing the ethyl lactate.

### Example A-2 (EA-2)

The procedures for producing ethyl lactate in this example were similar to those described in EA-1, except that the esterification reaction was carried out for 3 hours.

### Example A-3 (EA-3)

The procedures for producing ethyl lactate in this example were similar to those described in EA-1, except that the esterification reaction was carried out for 6 hours.

### Examples B-1 to B-3, C-1 to C-3, D-1 to D-3, E-1 to E-3, and F-1 to F-3, and Comparative Examples A-1 to A-3, B-1 to B-3, and C-1 to C-3

The procedures for producing ethyl lactate in each of Examples B-1 to B-3 (EB-1 to EB-3), C-1 to C-3 (EC-1 to EC-3), D-1 to D-3 (ED-1 to ED-3), E-1 to E-3 (EE-1 to EE-3), and F-1 to F-3 (EF-1 to EF-3), and Comparative Examples A-1 to A-3 (CEA-1 to CEA-3), B1 to B3 (CEB-1 to CEB-3), and C1 to C3 (CEC-1 to CEC-3) were similar to those described in EA-1, except that the type and amount of the antimony catalyst, and the conditions for carrying out the esterification reaction were varied as shown in Table 1.

### Determination of Weight Ratio of Antimony Catalyst to Lactic Acid

The weight ratio of the antimony catalyst to the lactic acid in each of the examples and comparative examples was calculated using the following Equation (I):

$$r = a / ( {l}^{′} \times {d}^{′} \times p ) \qquad ( I )$$

where r=weight ratio of antimony catalyst to lactic acid
   a=amount of antimony catalyst (g)
   l′=amount of lactic acid (mL)
d′=density of lactic acid (i.e., 1.209 g/mL)
p=purity of lactic acid (i.e., 90%)

### Determination of Yield of Ethyl Lactate:

The filtrate obtained in each of the above-mentioned examples and comparative examples was diluted with anhydrous ethanol, followed by adding cyclohexanol as an internal standard, so as to obtain a test sample. The test sample was then placed in a gas chromatography-flame ionization detector (GC-FID) (Zef Scientific, Inc.; Model: YL6500 GC) equipped with a gas chromatography column (Agilent; Model: DB-5 Capillary GC column, size: 30 m×0.25 mm×0.25 μm), followed by analysis to obtain an area ratio of the ethyl lactate to the cyclohexanol. Next, the number of moles of the ethyl lactate was calculated from the area ratio thus obtained and the number of moles of the cyclohexanol.

Thereafter, the yield of the ethyl lactate in each of the examples and comparative examples, which was obtained after the esterification reaction, was calculated using the following Equation (II):

$$Y = ( E / L ) \times 100 \% \qquad ( II )$$

where Y=yield of ethyl lactate
   E=number of moles of ethyl lactate
   L=number of moles of lactic acid

TABLE 1

| | Antimony catalyst | | | | | |
|---|---|---|---|---|---|---|
| | | Weight | | | | |
| | | ratio of | | | | |
| | | antimony | | | | Ethyl |
| | | catalyst | Esterification reaction | | | lactate |
| | | to lactic | Temperature | Pressure | Time | Yield |
| | Type | acid | (° C.) | (atm) | (hour(s)) | (%) |
| EA-1 | Sb₂O₃ | 0.03 | 80 | 1 | 1 | 39.4 |
| EA-2 | | | | | 3 | 50.5 |
| EA-3 | | | | | 6 | 63.4 |
| EB-1 | Sb₂O₅•nH₂O | 0.03 | 80 | 1 | 1 | 22.4 |
| EB-2 | | | | | 3 | 41.1 |
| EB-3 | | | | | 6 | 45.8 |
| EC-1 | SbCl₃ | 0.03 | 80 | 1 | 1 | 62.3 |
| EC-2 | | | | | 3 | 58.6 |
| EC-3 | | | | | 6 | 60.8 |
| ED-1 | Sb₂O₃ | 0.01 | 80 | 1 | 1 | 22.0 |
| ED-2 | | | | | 3 | 31.6 |
| ED-3 | | | | | 6 | 53.6 |
| EE-1 | Sb₂O₃ | 0.06 | 80 | 1 | 1 | 41.1 |
| EE-2 | | | | | 3 | 37.2 |
| EE-3 | | | | | 6 | 54.5 |
| EF-1 | Sb₂O₃ | 0.03 | 88 | 1 | 1 | 39.5 |
| EF-2 | | | | | 3 | 55.2 |
| EF-3 | | | | | 6 | 50.9 |
| CEA-1 | Sb₂O₃ | 0.03 | 25 | 1 | 1 | 0 |
| CEA-2 | | | | | 3 | 0 |
| CEA-3 | | | | | 6 | 19.4 |
| CEB-1 | Sb₂O₃ | 0.03 | 55 | 1 | 1 | 0 |
| CEB-2 | | | | | 3 | 0 |
| CEB-3 | | | | | 6 | 38.8 |
| CEC-1 | Sb₂O₃ | 0.03 | 80 | 0.1 | 1 | 24.1 |
| CEC-2 | | | | | 3 | 0 |
| CEC-3 | | | | | 6 | 0 |

Referring to Table 1, by virtue of using the antimony catalyst and allowing the esterification reaction to be carried out at the temperature ranging from 80° C. to 88° C. and under the pressure of 1 atm, in each of EA-1, EB-1, EC-1, ED-1, EE-1, and EF-1, the yield of the ethyl lactate obtained after 1 hour of the esterification reaction ranged from 22.0% to 62.3%; in each of EA-2, EB-2, EC-2, ED-2, EE-2, and EF-2, the yield of the ethyl lactate obtained after 3 hours of the esterification reaction ranged from 31.6% to 58.6%; and in each of EA-3, EB-3, EC-3, ED-3, EE-3, and EF-3, the yield of the ethyl lactate obtained after 6 hours of the esterification reaction ranged from 45.8% to 63.4%.

Additionally, the yield of the ethyl lactate obtained in each of CEA-1 after 1 hour of the esterification reaction and CEA-2 after 3 hours of the esterification reaction was 0%, and the yield of the ethyl lactate obtained in CEA-3 after 6 hours of the esterification reaction was merely 19.4%. The yield of the ethyl lactate obtained in each of CEB-1 after 1 hour of the esterification reaction and CEB-2 after 3 hours of the esterification reaction was 0%, and the yield of the ethyl lactate obtained in CEB-3 after 6 hours of the esterification reaction was merely 38.8%. Moreover, although the yield of the ethyl lactate obtained in CEC-1 after 1 hour of the esterification reaction was 24.1%, the yield of the ethyl lactate obtained in each of CEC-2 after 3 hours of the esterification reaction and CEC-3 after 6 hours of the esterification reaction dropped to 0%. These results show that in the respective one of the methods of EA-1 to EA-3, EB-1 to EB-3, EC-1 to EC-3, ED-1 to ED-3, and EF-1 to EF-3, by virtue of conducting the esterification reaction at the temperature of 80° C. or 88° C. and under the pressure of 1 atm, the yield of the ethyl lactate thus obtained is greatly improved compared with the yield of the ethyl lactate obtained in each of CEA-1 to CEA-3, CEB-1 to CEB-3, and CEC-1 to CEC-3, in which the esterification reaction was carried out at room temperature (25° C.), 55° C., or under the pressure of 0.1 atm.

In summary, in the method for producing ethyl lactate of the present disclosure, by selecting certain types of the antimony catalyst (i.e., antimony trioxide, antimony pentoxide, antimony trichloride, and combinations thereof) having a good stability, and by setting certain conditions for conducting the esterification reaction (i.e., the temperature ranging from 80° C. to 88° C. and the pressure of 1 atm), the antimony catalyst exhibits a good catalytic effect on the esterification reaction of lactic acid and ethanol. Additionally, the antimony catalyst has an advantage of being cheap. Therefore, the method of the present disclosure enables the ethyl lactate to be produced effectively and in a low-cost manner, thereby achieving the purpose of the invention.

In addition, because mild conditions are set for conducting the esterification reaction, applying the method for producing ethyl lactate of the present disclosure to industrial manufacturing not only makes the preparation of ethyl lactate relatively safe, but also results in a reduction in energy consumption during preparation of ethyl lactate, and is simple in operation.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for producing ethyl lactate, comprising:
subjecting lactic acid and ethanol to an esterification reaction in the presence of an antimony catalyst, so as to obtain ethyl lactate, the antimony catalyst being selected from the group consisting of antimony trioxide, antimony pentoxide, antimony trichloride, and combinations thereof, the esterification reaction being carried out at a temperature ranging from 80° C. to 88° C. and under a pressure of 1 atm.

2. The method as claimed in claim 1, wherein a weight ratio of the antimony catalyst to the lactic acid ranges from 0.01 to 0.06 in decimal form.

3. The method as claimed in claim 1, wherein the lactic acid has a purity of not lower than 90%.

4. The method as claimed in claim 1, wherein a volume ratio of the ethanol to the lactic acid ranges from 1.41 to 5.64 in decimal form.

5. The method as claimed in claim 4, wherein the lactic acid is present in an amount ranging from 5 parts by volume to 10 parts by volume, and the ethanol is present in an amount ranging from 14.1 parts by volume to 28.2 parts by volume.

6. The method as claimed in claim 1, wherein the esterification reaction is carried out for a time period ranging from 3 hours to 6 hours.
